(19) United States
(12) Patent Application Publication
(10) Pub. No.: US 2018/0146950 A1
(43) Pub. Date: May 31, 2018

(54) ULTRASONIC PROBE

(71) Applicant: CANON KABUSHIKI KAISHA, Tokyo (JP)

(72) Inventors: Shinji Ohishi, Oyama-shi (JP); Koichi Sentoku, Tokyo (JP); Daisuke Iwase, Tokyo (JP); Toshinobu Tokita, Yokohama-shi (JP); Takuro Miyasato, Tokyo (JP); Shigeru Ichihara, Tokyo (JP)

(21) Appl. No.: 15/817,982

(22) Filed: Nov. 20, 2017

(30) Foreign Application Priority Data
Nov. 25, 2016 (JP) 2016-229309
Nov. 25, 2016 (JP) 2016-229310

(51) Int. Cl. A61B 8/00; A61B 8/13

(52) U.S. Cl. CPC A61B 8/4461; A61B 5/0095; A61B 8/4427; A61B 8/13

(57) ABSTRACT

An ultrasonic probe including a unit that changes a quantity of light emitted on a subject.

### BACKGROUND

### Field of the Disclosure

The present disclosure relates to a hand-held ultrasonic probe.

### Description of the Related Art

One of the methods for obtaining an optical characteristic value such as an absorption coefficient in a subject includes photoacoustic tomography (PAT) using ultrasonic waves. A device using the PAT (hereinafter referred to as a photoacoustic device) has at least a light source and a probe.

First, when pulsed light generated from the light source is emitted on the living body, the light propagates while diffusing within the subject. An optical absorber in the subject absorbs the propagating light and generates a photoacoustic wave (typically ultrasonic wave). It is possible to obtain an initial sound pressure distribution caused by the optical absorber in the subject by probing this photoacoustic wave, outputting a detection signal, and analyzing the detection signal. The sound pressure P of ultrasonic wave obtained from the optical absorber in the subject caused by optical absorption in PAT can be expressed by the following equation:

P=Γ·μₐ·Φ (1)

In the above equation (1), P represents the initial sound pressure. Γ represents the Gruneisen factor which is an elasticity characteristic value, and is obtained by dividing the product of the volume expansion factor β and the square of the sound speed c by the specific heat Cₚ. μₐ represents the absorption coefficient of the optical absorber, and Φ represents the quantity of light absorbed by the optical absorber. As can be understood from this equation, an absorption coefficient can be obtained by considering the quantity of light reaching an arbitrary position with respect to the initial sound pressure at that position. Since the absorption coefficient varies depending on the optical absorber, the distribution of optical absorbers constituting the subject, such as the distribution of blood vessels, can be known by obtaining the distribution of the absorption coefficients of the subject.

Japanese Patent Application Laid-Open No. 2016-49212 discusses a photoacoustic probe provided with an irradiation direction changing unit capable of changing the irradiation direction of light from a light irradiation unit, wherein the irradiation direction of the light source is changed when a region of interest (ROI) is designated on the scanning screen.

Japanese Patent Application Laid-Open No. 2016-49191 discusses a photoacoustic probe provided with a mechanism for tilting the optical axis of the light emitting element in synchronization with the inclination of the acoustic wave detection unit. More specifically, Japanese Patent Application Laid-Open No. 2016-49191 discusses a configuration in which light source units including light emitting elements are provided on both side surfaces of the acoustic wave detection unit, wherein the inclination of the optical axis of the light emitting element is changed by changing the inclination of the light source unit according to the inclination of the acoustic wave detection unit.

However, the present inventors have found a problem wherein, to change the irradiation angle on the operation screen while the operator (user) of the ultrasonic probe presses the ultrasonic probe on the subject, it is necessary to operate with a pointing device such as a mouse, which results in poor operability.

### SUMMARY

The present disclosure is directed to providing an ultrasonic probe capable of operation while allowing an operator (user) of the probe to easily change a light irradiation position, without effecting operability. The present inventors have found that a problem occurs in the photoacoustic probe discussed in Japanese Patent Application Laid-Open No. 2016-49212. More specifically, there occurs a problem that, in a case where the acoustic wave detection unit is greatly tilted or when a subject having a high curvature such as an arm is an observation target, one of the light source units is not in contact with the subject, and light from one light source unit fails to emit light on the subject, whereby the quantity of light emitted on the subject decreases.

The present disclosure is also directed to an ultrasonic probe capable of irradiating a subject with a sufficient quantity of light regardless of the contact state with the subject.

According to an aspect of the present disclosure, a hand-held ultrasonic probe includes a grip unit, a light irradiation unit, and an ultrasonic reception unit, wherein the light irradiation unit includes a change unit configured to change a light intensity distribution of light emitted on a subject, and a reception unit configured to receive an instruction from a user about a change in the light intensity distribution of the light from the light irradiation unit in a state in which the user grips the ultrasonic probe, and wherein the light irradiation unit obtains a signal for controlling the change unit based on the instruction from the user received by the reception unit, and drives the change unit using the signal to change the light intensity distribution of the light for the subject.

According to another aspect of the present disclosure, a hand-held ultrasonic probe includes a light emission unit configured to emit light on a subject, an ultrasonic reception unit configured to receive an ultrasonic wave generated by emission of the light on the subject, and an acquisition unit configured to acquire information about a contact state between the subject and the ultrasonic probe, wherein the light emission unit includes a plurality of light emission end portions of which output light quantities are independently controlled, and wherein light with light quantities controlled based on the information about the contact state is output from the plurality of light emission end portions.

According to the ultrasonic probe of the first aspect of the present disclosure, the light intensity distribution of the light emitted on the subject can easily be changed while the probe operator (user) grips the ultrasonic probe, whereby the operability is improved.

According to the ultrasonic probe of the second aspect of the present disclosure, the subject can be irradiated with light with sufficient light intensity regardless of the contact state with the subject.

Further features of the present disclosure will become apparent from the following description of exemplary embodiments with reference to the attached drawings.

### BRIEF DESCRIPTION OF THE DRAWINGS

FIG. 1 illustrates an ultrasonic probe according to an exemplary embodiment of the present disclosure.

FIGS. 2A and 2B are diagrams illustrating ultrasonic probe operation according to the exemplary embodiment of the present disclosure.

FIGS. 3A and 3B illustrate an ultrasonic probe according to the exemplary embodiment of the present disclosure (Example 1).

FIGS. 4A and 4B illustrate an ultrasonic probe according to the exemplary embodiment of the present disclosure (Example 2).

FIGS. 5A and 5B illustrate an ultrasonic probe according to the exemplary embodiment of the present disclosure (Example 3).

FIG. 6 illustrates an example of an image display unit according to the exemplary embodiment of the present disclosure.

FIG. 7 illustrates an ultrasonic probe device according to the exemplary embodiment of the present disclosure (Example 1) in which a light source is arranged outside.

FIG. 8 illustrates an ultrasonic probe according to another exemplary embodiment of the present disclosure.

FIG. 9 illustrates an ultrasonic probe according to the another exemplary embodiment of the present disclosure (Example 1).

FIGS. 10A and 10B are diagrams illustrating a problem of a conventional ultrasonic probe.

FIG. 11 illustrates an ultrasonic probe according to the another exemplary embodiment of the present disclosure (Example 1) in which a light source is arranged outside.

FIG. 12 illustrates an ultrasonic probe according to the another exemplary embodiment of the present disclosure (Example 2).

FIG. 13 illustrates the ultrasonic probe according to the another exemplary embodiment of the present disclosure (Example 2) with a subject having a large curvature.

FIGS. 14A and 14B illustrate an ultrasonic probe according to the another exemplary embodiment of the present disclosure (Example 3).

FIGS. 15A, 15B, and 15C are cross-sectional views of the ultrasonic probe according to the another exemplary embodiment of the present disclosure.

### DESCRIPTION OF THE EMBODIMENTS

In a first exemplary embodiment, an example of an ultrasonic probe according to a first aspect of the present disclosure will be described. Note that the configuration of a second exemplary embodiment described below may be added to the configuration described in the first exemplary embodiment.

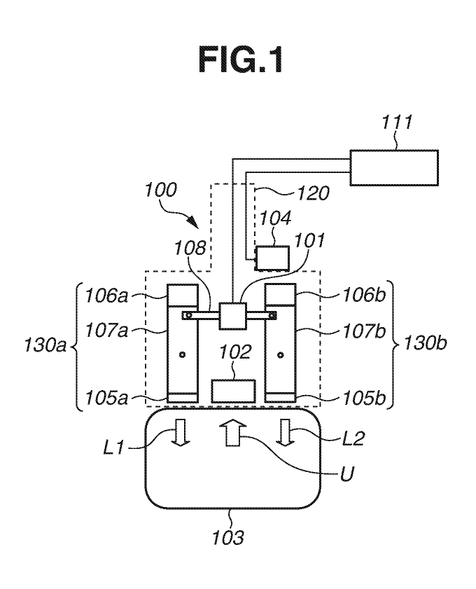

The ultrasonic probe according to an exemplary embodiment of the present disclosure is illustrated in FIG. 1, but the ultrasonic probe according to the exemplary embodiment of the present disclosure is only an example, and the present disclosure is not limited thereto. The ultrasonic probe 100 according to the present exemplary embodiment is a hand-held ultrasonic probe including a grip unit 120, light irradiation units 130a, 130b, and an ultrasonic reception unit 102. In this configuration, the light irradiation units 130a and 130b include light sources 106a and 106b, light guide paths 107a and 107b, and light emission end portions 105a and 105b respectively. However, as described below, if the light source is located outside the ultrasonic probe, the light irradiation unit 130 does not include a light source. Examples of light sources include a light emitting diode (LED), a laser diode (LD), and a solid-state laser. When an LED or an LD is used as the light source, it is desirable to obtain a large light output by using a light source array in which these light sources are arranged in an array. A compact light source array such as an LED array or an LD array is desirably included in the ultrasonic probe, in view of reducing the weight of the device and obtaining a sufficient light output.

Meanwhile, to obtain a larger light output, a solid laser such as an alexandrite laser, a titanium sapphire laser, or an optical parametric oscillator (OPO) laser is desirably used as a light source. When a solid laser is used as a light source, it is desirable to provide a wave guide unit for guiding light from an externally provided light source to the light emission end portion.

As illustrated in FIG. 1, the light sources 106a and 106b that generate light for irradiating a subject 103 are provided at the sides of the probe 100, so that the light is emitted from the light emission end portions 105a and 105b via the light guide paths 107a and 107b. Furthermore, an ultrasonic reception unit 102 is provided to receive an ultrasonic wave U generated from the subject 103 when the subject 103 is irradiated with light L1 and light L2.

A change unit 101 for changing the light intensity distribution of light and a reception unit 104 for receiving an instruction from the user concerning the change of the light intensity distribution of the light from the light irradiation unit 130 in a state where the user grips the ultrasonic probe are provided. Based on an instruction from the user received by the reception unit 104, a signal for controlling the change unit is obtained, and the change unit 101 is driven using the signal to change the light intensity distribution of the light emitted on the subject.

When the reception unit 104 has a contact sensor for acquiring information about the contact state between the subject 103 and the ultrasonic probe 100, an instruction from the user is given via the contact sensor. As the reception unit 104, a pressure sensor may be provided in the grip unit 120, and an instruction from the user may be given via the pressure sensor. Furthermore, as the reception unit 104, a switch unit may be provided in the ultrasonic probe 100, and an instruction from the user may be given via the switch unit. Alternatively, all of these three reception units 104 may be provided, or two of these three reception units 104 may be provided.

As the change unit 101 that changes the light intensity distribution of the light emitted on the subject 103, it is possible to use a unit for changing the irradiation position of the light emitted on the subject. The ultrasonic wave from the optical absorber near the surface of the subject 103 (shallow place) can be detected by setting the irradiation position of the light with respect to the subject 103 close to the ultrasonic reception unit 102. On the other hand, by setting the irradiation position of the light with respect to the subject 103 away from the ultrasonic reception unit 102, it is easy to cause the light to spread through the subject 103 and to detect the ultrasonic wave from the optical absorber in a deep place of the subject 103. This is because irradiation of light to a position far from the ultrasonic reception unit 102 can reduce the clutter noise occurring near the surface of the subject 103.

A unit for changing the irradiation position of light may be a unit that moves on the surface of the subject 103 in parallel therewith, or the irradiation position can be changed by changing the angle of light emitted on the subject. In the following, an example will be described in a case where a unit for changing the irradiation angle is used as the change unit 101.

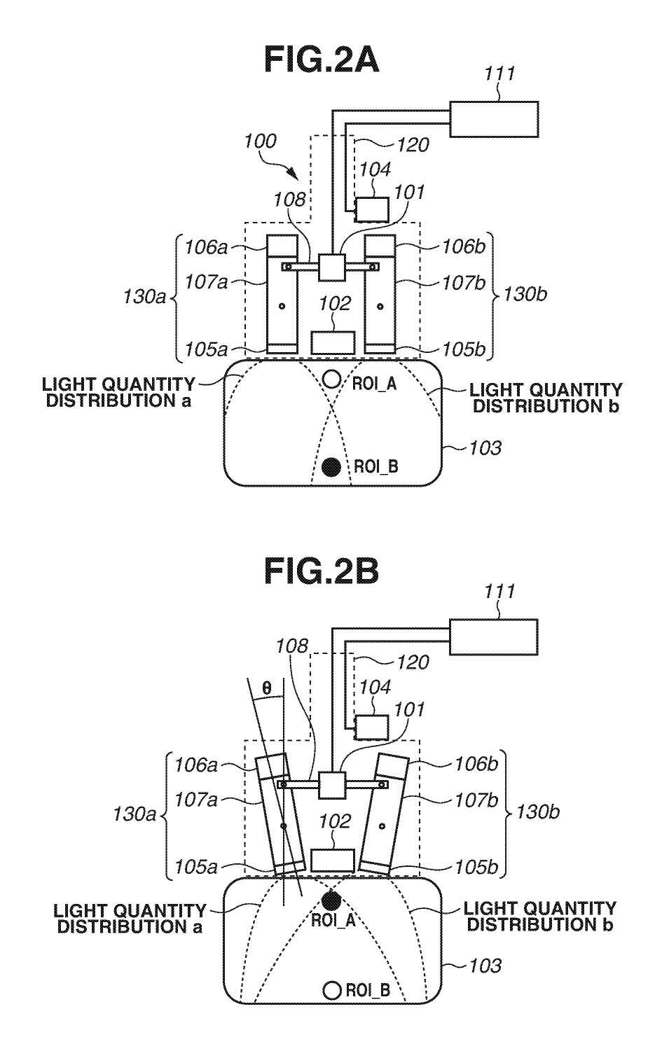

FIGS. 2A and 2B illustrate an example of a light intensity distribution when light is emitted from the light emission end portions 105a and 105b of the ultrasonic probe 100 toward the subject 103. In FIGS. 2A and 2B, the inner side region indicated by a broken line indicates the light intensity distribution from each light source. When light is emitted in a state where the angle of the optical axis is kept perpendicular (zero) with respect to the subject 103 (FIG. 2A), light beams from the two light sources reach a deep area in an overlapping manner, and therefore, a photoacoustic wave can be detected from a deep region of interest (ROI) (ROI_B). Since it is difficult for light to be emitted on the subject near the ultrasonic reception unit 102, a pseudo signal (clutter noise) from near the surface of the subject is hardly detected.

However, since the light does not reach a shallow part (ROI_A) immediately below the ultrasonic reception unit 102, the acoustic wave becomes weak, and it is easy to detect the photoacoustic wave from the deep ROI (ROI_B).

On the other hand, when the angle of the optical axis from each of the light irradiation units 130a and 130b with respect to the subject 103 is 8 (theta) as illustrated in FIG. 2B, the light also reaches the shallow ROI (ROI_A) immediately below the ultrasonic reception unit 102. Therefore, it is possible to detect a photoacoustic wave from the shallow ROI (ROI_A). In this case, the light reaches the deep ROI (ROI_B), but the light reaching the deep place is weak and the signal from the shallow ROI (ROI_A) becomes dominant. In this way, by switching the light intensity distribution, light can be emitted effectively to the shallow ROI and the deep ROI.

Since the user can change the light intensity distribution of light while gripping the grip unit 120 as described above, the operability is improved.

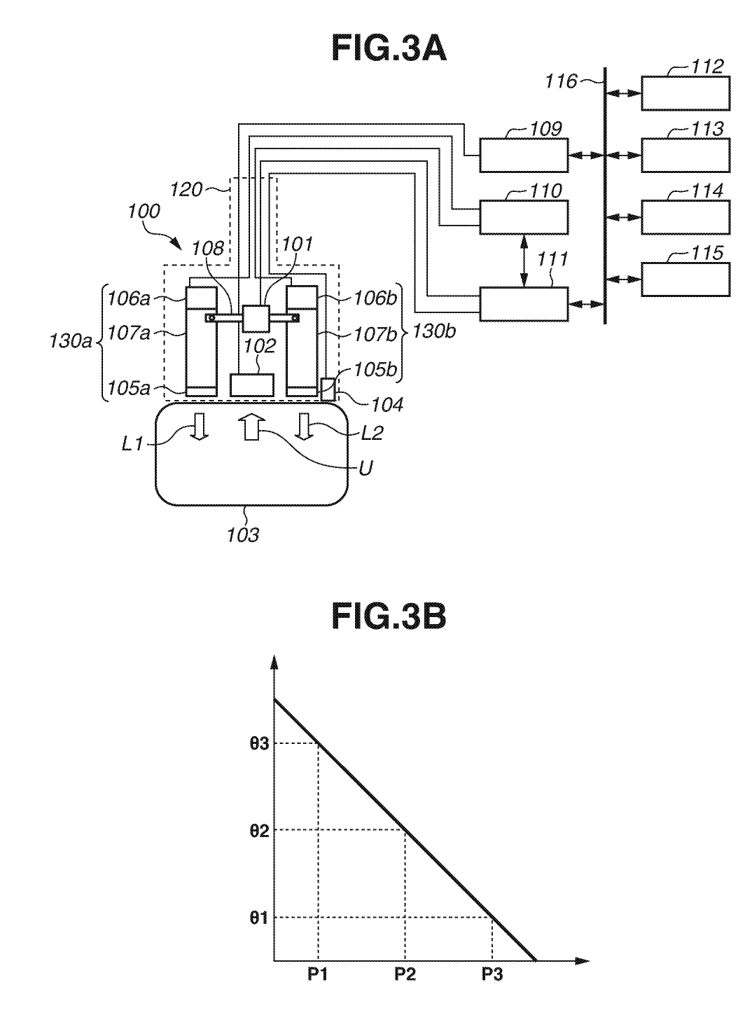

The hand-held ultrasonic probe device according to Example 1 is illustrated in FIGS. 3A and 3B. In the present exemplary embodiment, a light source (LED array, LD array) 106 is provided on each side of the probe, and light emitted from the light source is guided to the light emission end portion 105 by the light guide path 107. After the ultrasonic probe 100 is appropriately pressed on the subject 103 by the user, light is emitted toward the subject 103. The light source 106 controls the ON/OFF state and the light intensity of the irradiation by a light source control unit 110. The acoustic wave generated in the subject receiving the light is received by the ultrasonic reception unit 102, and then sent to a signal processing unit 109, whereby subject information is obtained. In the case of a device using the photoacoustic effect, the acquired subject information indicates a generation source distribution of the acoustic waves generated by light irradiation, an initial sound pressure distribution in the subject, a light energy absorption density distribution and an absorption coefficient distribution derived from the initial sound pressure distribution, and a concentration distribution of substances constituting tissues. The concentration distribution of substances is, for example, an oxygen saturation distribution, a total hemoglobin concentration distribution, and an oxidized/reduced hemoglobin concentration distribution.

The ultrasonic probe device further includes an operation unit 114 for a user (mainly an examiner such as a medical worker) to input instructions such as start of imaging, and parameters required for imaging, to the device, and an image configuration unit 113 that images the obtained subject information. The ultrasonic probe device also includes a display unit 112 that displays a user interface (UI) for operating the generated image and the device.

The device further includes a control processor 111 that receives various operations of the user via the operation unit 114, generates control information required for generating target subject information, and controls each function via a system bus 116. The device further includes a storage unit 115 that stores the acquired photoacoustic wave digital signal, generated image, and other operation-related information. The details of each unit will be described below.

### (Light Source)

From the light source 106 according to the present exemplary embodiment, pulsed light of a wavelength absorbed by a specific component of the components constituting the living body is emitted. It is desirable that the wavelength used in the present exemplary embodiment be a wavelength allowing light to propagate to the inside of the subject. More specifically, when the subject is a living body, the wavelength used in the present exemplary embodiment is 600 nm or more and 1100 nm or less. In order to efficiently generate a photoacoustic wave, the pulse width is desirably about 10 to 100 nanoseconds. As a light source, a laser capable of obtaining a large output is desirable, but an LED, a flash lamp, or the like can be used instead of the laser. As the laser, various lasers such as a solid laser, a gas laser, a dye laser, and a semiconductor laser can be used. The time point, waveform, intensity, and the like of irradiation are controlled by the light source control unit. This light source control unit may be integrated with the light source. The light source unit may be provided separately from the photoacoustic device according to the present exemplary embodiment.

The light source according to the present exemplary embodiment may be a light source capable of emitting light of a plurality of wavelengths.

### (Ultrasonic Reception Unit)

A detection element is arranged in the ultrasonic reception unit 102 according to the present exemplary embodiment, and the detection element outputs a detection signal by detecting a photoacoustic wave occurring in the inside of the living body and on the surface of the living body by the pulsed light. The detection element is for converting a photoacoustic wave into an electric signal. Any detection element may be used as long as it can detect a photoacoustic wave, such as a detection element using a piezoelectric phenomenon, a detection element using a resonance of light, and a detection element using a change of an electrostatic capacity. A piezo micromachined ultrasonic transducer (PMUT) is an example of piezoelectric transducers using a piezoelectric phenomenon. A capacitive micromachined ultrasonic transducer (CMUT) is an example of electrostatic capacity type transducers using changes in electrostatic capacity. The CMUT is more desirable as a detection element because it can detect a photoacoustic wave in a wide frequency band.

In order to obtain a high-resolution photoacoustic image, it is desirable to arrange a plurality of detection elements in two dimensions or three dimensions to perform scanning. A reflection film such as a gold film may be provided on the surface of the probe to return the light reflected from the surface of the subject or the holding part, or the light scattered in the inside of the subject and coming out of the subject, to the subject again.

### (Signal Processing Unit)

The signal processing unit 109 according to the present exemplary embodiment amplifies the photoacoustic wave signal generated by the ultrasonic reception unit 102 and converts the signal into a photoacoustic wave digital signal which is a digital signal. The signal processing unit 109 according to the present exemplary embodiment includes a signal amplification unit (not illustrated) that amplifies the analog signal generated by the ultrasonic reception unit 102, and an analog-to-digital (A/D) conversion unit (not illustrated) that converts the analog signal into a digital signal.

Furthermore, for the photoacoustic wave digital signal, the signal processing unit 109 according to the present exemplary embodiment performs correction of the sensitivity variation of the ultrasonic reception unit 102 and supplementation processing of the transducer physically or electrically damaged. Further, the signal processing unit 109 can also perform integration processing for noise reduction and the like. The photoacoustic signal obtained by detecting the photoacoustic wave emitted by a light absorption substance inside of the subject 103 is generally a weak signal. By applying integration averaging processing to the photoacoustic wave signals obtained repeatedly at the same position of the subject 103 by the integration processing, system noise can be reduced and the signal-to-noise (S/N) ratio of the photoacoustic wave signal can be improved.

### (Control Processor)

The control processor 111 according to the present exemplary embodiment runs an operating system (OS) to control and manage basic resources in the program operation, reads the program code stored in the storage unit 115, and executes the functions described below. The control processor 111 receives event notifications generated by various operations such as start of imaging from the user via the operation unit 114, manages acquisition operation of the subject information, and controls each piece of hardware via the system bus 116. The control processor 111 further commands the light source control unit 110 to control the light source 106 required for generating the subject information of interest. An example of the control processor is a central processing unit (CPU).

### (Operation Unit)

The operation unit 114 according to the present exemplary embodiment is an input device for the user to perform parameter setting related to imaging of, for example, the visualization range of the subject information, instruction to start imaging, and other image processing operations related to images. In general, the operation unit 114 according to the present exemplary embodiment is constituted by a mouse, a keyboard, a touch panel, and the like, and performs event notification to the software such as the OS operating on the control processor 111 according to the operation of the user.

### (Image Configuration Unit)

Based on the acquired photoacoustic wave digital signal, the image configuration unit 113 according to the present exemplary embodiment converts tissue information within the subject into an image and constructs a display image such as an arbitrary tomographic image of the photoacoustic wave image. Various correction processes such as luminance correction, distortion correction, and ROI cropping are applied to the constructed image to construct more desirable information for diagnosis. According to the operation of the user via the operation unit 114, parameters related to the configuration of the photoacoustic wave image are adjusted, and display images are adjusted. The photoacoustic wave image is obtained by performing image reconstruction processing on the digital signal of the three-dimensional photoacoustic wave generated from the ultrasonic reception unit 102. The photoacoustic wave image can visualize a characteristic distribution such as acoustic impedance and subject information such as an optical characteristic value distribution. For example, back projection in time domain or Fourier domain commonly used in tomography technology, or phasing addition processing is used for the image reconstruction processing. The image configuration unit 113 is generally constructed using a graphics processing unit (GPU) having a high-performance arithmetic operation processing function and a graphic display function. This can shorten the time taken to execute the image reconstruction processing and to configure a display image.

### (Display Unit)

The display unit 112 according to the present exemplary embodiment displays a photoacoustic wave image generated by the image configuration unit 113 and a UI for operating an image and the device. For example, a liquid crystal display is used, but any type of display such as organic electro luminescence (EL) may be used.

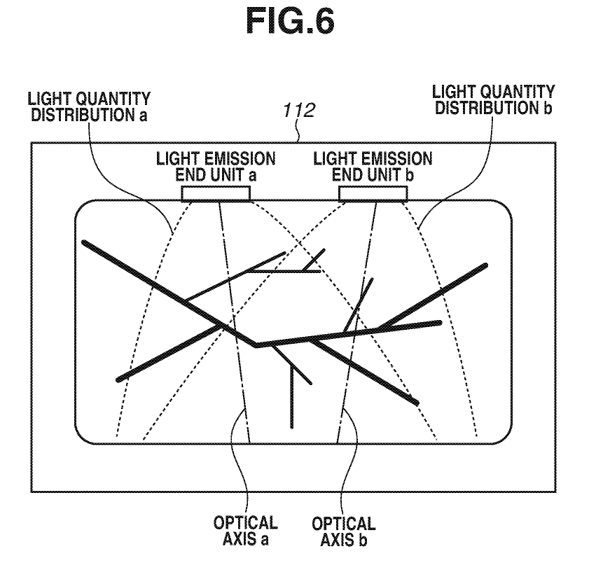

FIG. 6 illustrates a display example of a photoacoustic image. By displaying the light irradiation area illustrated in FIGS. 2A and 2B (broken line in FIGS. 2A and 2B) and the inclination of the optical axis (one-dot chain line in FIG. 6) in an overlapping manner on the photoacoustic image (thick solid line in FIG. 6), the probe operator operates the reception unit provided in the probe and decides the ROI while looking at the screen. Information such as the optical axis angle may be displayed on the display unit 112 numerically.

### (Storage Unit)

The storage unit 115 according to the present exemplary embodiment includes a memory required for the control processor 111 to operate, and a memory temporarily holding data in the subject information acquisition operation. Furthermore, the storage unit 115 includes a storage medium such as a hard disk that stores and holds the generated photoacoustic wave image, relevant subject information, diagnostic information, measurement conditions such as optical axis angle, and the like. The storage unit 115 stores the program code of software that realizes the function described below.

### (Subject (Optical Absorber))

The subject (optical absorber) does not form part of the ultrasonic probe according to the present exemplary embodiment, but the subject (optical absorber) will be described below. The main purposes of the ultrasonic probe according to the present exemplary embodiment using the photoacoustic effect include imaging blood vessels, diagnosis of human and animal malignant tumor and vascular disease, and follow-up of chemotherapy. The optical absorber inside of the subject has an absorption coefficient relatively high within the subject depending on the wavelength of the light used. More specifically, the optical absorber inside of the subject may be water, fat, protein, oxidized hemoglobin, reduced hemoglobin, and the like.

Subsequently, the operation for changing the light intensity distribution will be described. As illustrated in FIG. 3A, as the reception unit 104 for receiving an instruction from the user regarding the change of the light intensity distribution, a pressure sensor for detecting the contact pressure with the subject is provided at the tip of the probe. An example of the pressure sensor is a load sensor such as a load cell. By placing a plurality of sensors at the tip of the probe and performing averaging processing, accuracy of detecting the contact pressure can be improved. The user can designate the ROI by adjusting the contact pressure (pressing force) of the probe 100 against the subject 103.

The information about the contact pressure from the reception unit 104 is taken into the control processor 111 and a signal for controlling the angle of the light from the light emission end portion 105 is sent to an optical axis angle change unit 101 according to the contact pressure, so that the angle of the optical axis is controlled by a transmission unit 108. In this case, examples of the optical axis angle change unit 101 include a drive mechanism with a rotary motor and a feed screw, a drive mechanism with a rotary motor and a cam, and a direct mechanism using a linear motor. The optical axis angle change unit 101 and the light source unit (the light source 106, the light guide path 107, and the light emission end portion 105) are connected by the transmission unit 108 such as a rod, and the angle of the optical axis is controlled.

An example of the relationship between the contact pressure and the optical axis angle is illustrated in FIG. 3B. In this example, the control processor 111 performs control so that, when the contact pressure is small (P1), the optical axis angle is large (Θ3), and when the contact pressure is large (P3), the optical axis angle is small (Θ1). When the user wishes to see a deep ROI, the user can designate the ROI with an intuitive and easy operation like strongly pushing the subject. The relationship between the contact pressure and the optical axis angle can be not only a linear relational expression but also a nonlinear relational expression, and an optimum relational expression can be set according to the state (e.g., hardness) of the subject, for example. Further, the usability is improved by providing a function of fixing (locking) to an optical axis angle determined with a certain contact pressure, a function of prohibiting the optical axis angle adjustment function from the beginning, and the like.

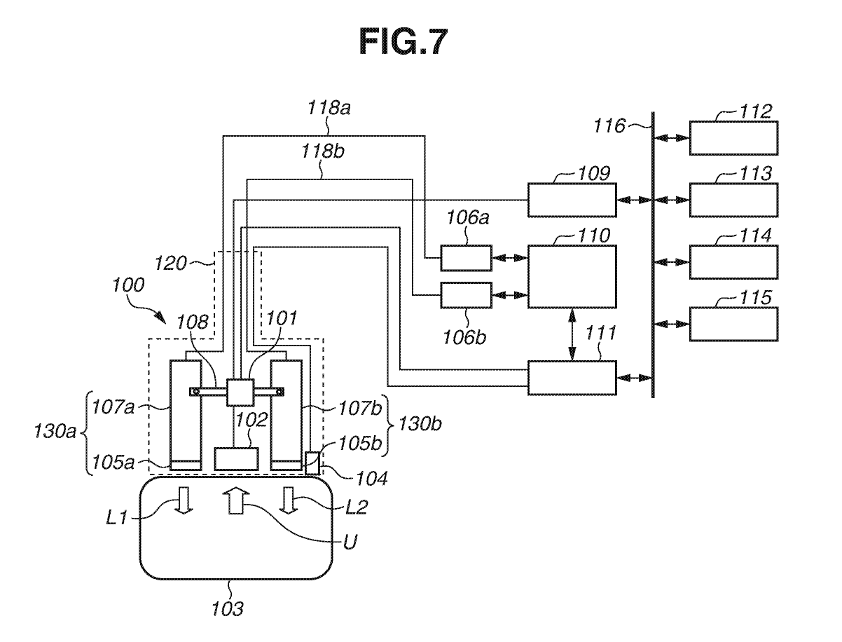

FIG. 7 illustrates an ultrasonic probe provided with a wave guide unit 108 (e.g., an optical fiber) for guiding light from the externally provided light source 106 to the light emission end portion. There is an advantage in that, for example, when the light source is provided externally, keeping the heat source away from the probe 100 makes thermal design easier, and maintenance of the light source becomes easier.

According to this configuration, the user of the probe 100 can change the angle of the optical axis with a simple operation that adjusts the indentation quantity (contact pressure) to the subject while gripping the probe, and as a result, the ROI can be designated by changing the light intensity distribution.

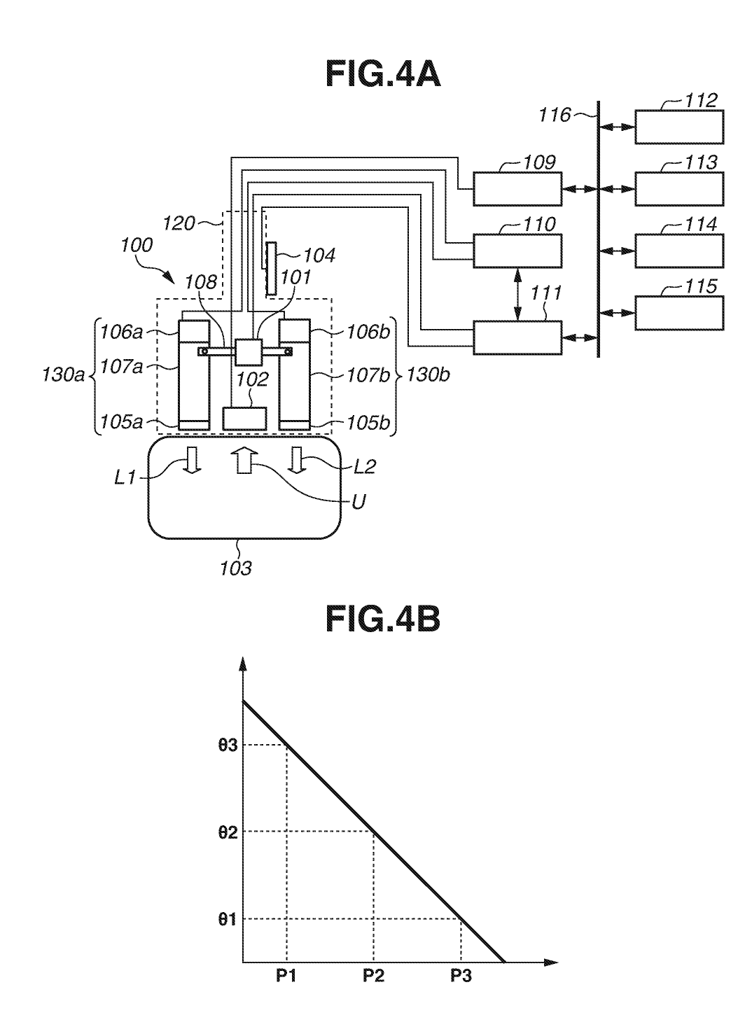

The hand-held ultrasonic probe device according to Example 2 is illustrated in FIGS. 4A and 4B. Subsequently, operation for changing the light intensity distribution will be described. As illustrated in FIG. 4A, a pressure sensor for detecting the user's gripping force (grasping force) is provided in a probe grip (grip) as the reception unit 104 for receiving an instruction from the user regarding the change of the light intensity distribution. An example of the pressure sensor is a load sensor such as a load cell. The user can designate the ROI by adjusting the gripping force (grasping force) on the probe 100.

The information about the gripping force from the reception unit 104 is taken into the control processor 111 and a signal for controlling the angle of the light from the light emission end portion 105 is sent to the optical axis angle change unit 101 according to the gripping force, so that the angle of the optical axis is controlled by the transmission unit 108. In this case, examples of the optical axis angle change unit 101 include a drive mechanism with a rotary motor and a feed screw, a drive mechanism with a rotary motor and a cam, and a direct mechanism using a linear motor. The optical axis angle change unit 101 and the light source unit (the light source 106, the light guide path 107, and the light emission end portion 105) are connected by the transmission unit 108 such as a rod, and the angle of the optical axis is controlled.

An example of the relationship between the gripping force and the optical axis angle is illustrated in FIG. 4B. In this example, the control processor 111 performs control so that, when the gripping force is small (P1), the optical axis angle is large (Θ3), and when the gripping force is large (P3), the optical axis angle is small (Θ1). When the user wishes to see a deep ROI, the user can designate the ROI with an intuitive and simple operation such as increasing the gripping force (grasping force).

The relationship between the gripping force and the optical axis angle can be not only a linear relational expression but also a nonlinear relational expression, and an optimum relational expression can be set according to the state (e.g., hardness) of the subject, for example. Further, the usability is improved by providing a function of fixing (locking) to an optical axis angle determined with a certain gripping force, a function of prohibiting the optical axis angle adjustment function from the beginning, and the like.

According to this configuration, the user can change the angle of the optical axis with a simple operation of adjusting the gripping force while gripping the probe, and as a result, the ROI can be designated by changing the light intensity distribution. Since it is not necessary to strongly press the probe against the subject, there is an advantage in that there is no pain or discomfort to the subject (examinee).

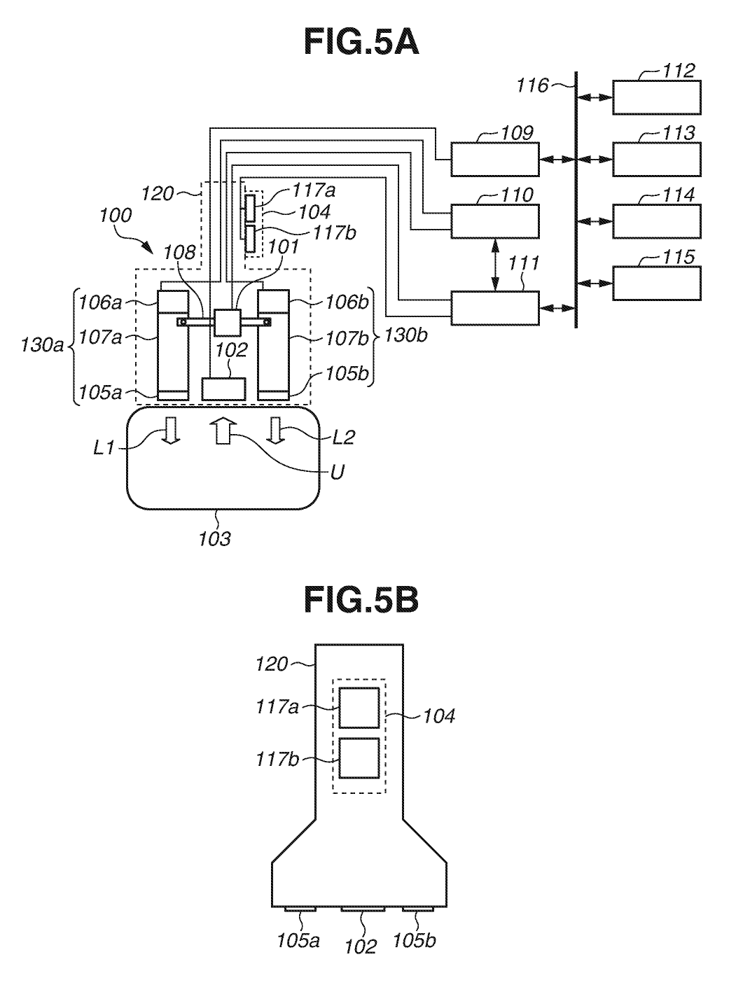

The hand-held ultrasonic probe device according to Example 3 is illustrated in FIGS. 5A and 5B. Subsequently, operation for changing the light intensity distribution will be described. As illustrated in FIG. 5B, for example, a switch unit (hereinafter simply referred to as switch) 117 is provided in the grip unit 120 of the probe as the reception unit 104 for receiving an instruction from the operator concerning change of the light intensity distribution. Examples of the switch include a push button switch, a slide switch, and a touch panel switch. As illustrated in FIG. 5B, the arrangement example of the switch 117 may be such that two switches may be provided, i.e., a switch 117a for designating a shallow ROI and a switch 117b for designating a deep ROI. The user can designate the ROI with an intuitive and simple operation such as operating the switch 117b when wishing to see a deep ROI and operating the switch 117a when wishing to see a shallow ROI.

The information about the switch from the reception unit 104 is captured by the control processor 111, and a signal for controlling the angle of the light from the light emission end portion 105 is sent to the optical axis angle change unit 101 according to the switch information, so that the angle of the optical axis is controlled by the transmission unit 108. In this case, examples of the optical axis angle change unit 101 include a drive mechanism with a rotary motor and a feed screw, a drive mechanism with a rotary motor and a cam, and a direct mechanism using a linear motor. The optical axis angle change unit 101 and the light source unit (the light source 106, the light guide path 107, and the light emission end portion 105) are connected by the transmission unit 108, and the angle of the optical axis is controlled.

According to this configuration, the operator of the probe 100 can change the angle of the optical axis with a simple operation of the switch while grasping the probe, and as a result, the ROI can be designated by changing the light intensity distribution. Further, in the present exemplary embodiment, there is no need to strongly press the probe against the subject, so there is an advantage in that no pain or discomfort is given to the examinee (subject).

The description of the reference numerals in the description of the first exemplary embodiment and FIGS. 1 to 7 will be given below.
100 ultrasonic probe
101 change unit
102 ultrasonic reception unit
103 subject
104 reception unit
105a, 105b light emission end portion
106a, 106b light source
107a, 107b light guide path
108 transmission unit
109 signal processing unit
110 light source control unit
111 control processor
112 display unit
113 image configuration unit
114 operation unit
115 storage unit
116 system bus
117a, 117b switch
118a, 118b wave guide unit
120 grip unit
130a, 130b light irradiation unit

In the second exemplary embodiment, an example of an ultrasonic probe according to a second aspect of the present disclosure will be described. It should be noted that the configuration described in the second exemplary embodiment may further include the configuration of the first exemplary embodiment described above.

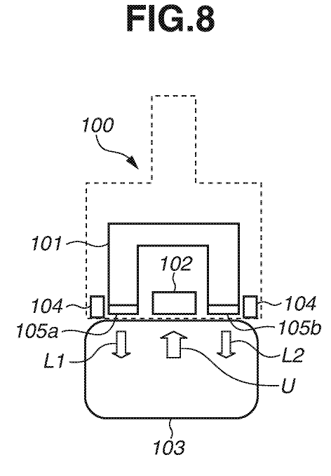

In the following description, an ultrasonic probe according to an exemplary embodiment of the present disclosure is illustrated in FIG. 8, but the ultrasonic probe according to the exemplary embodiment of the present disclosure is only an example, and the present disclosure is not limited thereto. The ultrasonic probe 100 according to the present exemplary embodiment includes a light emission unit 101 for emitting light to the subject 103, and an ultrasonic reception unit 102 for receiving ultrasonic waves generated by emission of light to the subject 103. Furthermore, an acquisition unit 104 for acquiring information about the contact state between the subject 103 and the ultrasonic probe 100 is provided, and the light emission unit 101 includes a plurality of light emission end portions 105a and 105b of which output light quantities (light intensities) are independently controlled. The plurality of light emission end portions 105a and 105b is configured to output the light with a light quantity controlled based on the information about the contact state acquired by the acquisition unit 104.

Based on the information about the contact state between the subject 103 and the ultrasonic probe 100, the quantity of light to be emitted on the subject 103 can be adjusted and thus the subject can be irradiated with light of a sufficient light quantity regardless of the contact state with the subject 103. For example, if the ultrasonic probe 100 is inclined with respect to the subject 103, and a part of the ultrasonic probe 100 is in a non-contact state, the quantity of light emitted on the subject 103 decreases, and sufficient ultrasonic waves for obtaining the information about the subject may not be generated in some cases. Therefore, control is performed to, for example, increase the light quantity of the light emitted from the light emission end portions 105a and 105b based on information about the contact state, such as that the ultrasonic probe 100 is in a non-contact state with the subject 103, or the contact is not sufficient. As a result, the quantity of light emitted on the subject 103 can be sufficient, and enough ultrasonic waves can be generated and detected to obtain the information about the subject.

In the ultrasonic probe 100 according to the exemplary embodiment of the present disclosure, the information about the contact state is not only the information about whether the ultrasonic probe is in the contact state or the non-contact state with the subject 103 but also a concept including the strength (degree) of contact.

Examples of the acquisition unit 104 for acquiring information about the contact state include a sensor (load sensor) for detecting a contact pressure when the subject 103 is pressed against the ultrasonic probe 100, and a displacement sensor for detecting a displacement caused by a change in the contact strength. The acquisition unit 104 may be a gradient sensor that acquires information about a change in the inclination of the ultrasonic probe 100 that is caused as the ultrasonic probe 100 changes from a state in which the entire surface of the ultrasonic probe 100 is in contact with the subject 103 to a state in which the entire surface of the ultrasonic probe 100 is in a non-contact state. In FIG. 8, two acquisition units for acquiring information about the contact state are illustrated, but one acquisition unit may be provided, or three or more acquisition units may be provided.

The light emission unit 101 itself may have a light source that generates light, or may guide light from a separately provided light source. The light emission unit 101 may be provided in a casing of the ultrasonic probe 100 or may be detachable with respect to a casing having the ultrasonic reception unit 102. Examples of light sources include an LED, an LD, and a solid-state laser. When the LED or the LD is used as the light source, it is desirable to obtain a high light output by using a light source array in which these light sources are arranged like an array. If a compact light source array such as an LED array or an LD array is built in the ultrasonic probe, this is desirable from the viewpoint of obtaining a sufficient light output with a light weight.

Meanwhile, to obtain a larger light output, it is desirable to use a solid laser such as an alexandrite laser, a titanium sapphire laser, or an OPO laser as a light source. When a solid laser is used as the light source, it is desirable to provide a wave guide unit for guiding light from the externally provided light source to the light emission end portion.

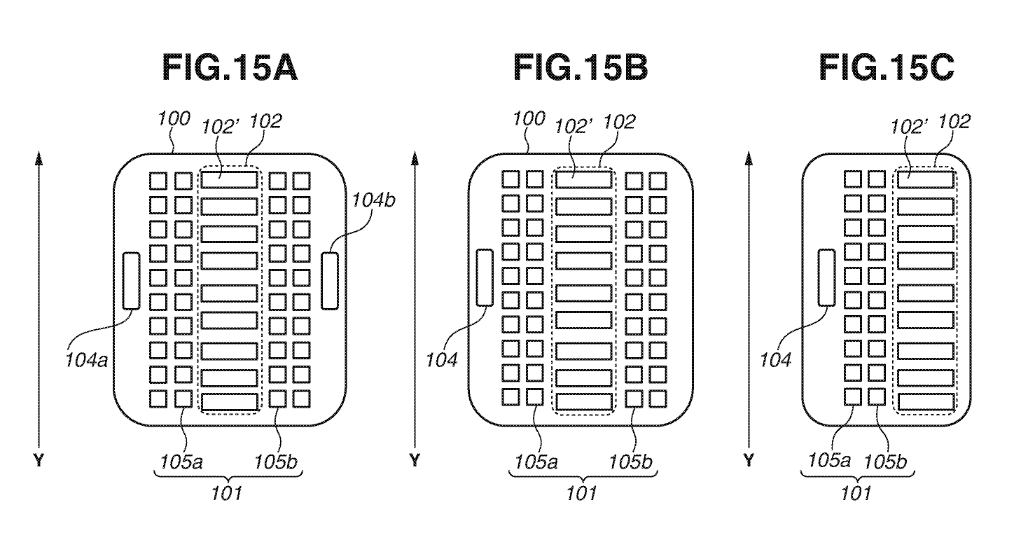

In this case, an example of the arrangement of the light emission end portion 105, the ultrasonic reception unit 102, and the acquisition unit 104 according to an exemplary embodiment of the present disclosure is illustrated in FIGS. 15A, 15B, and 15C. FIGS. 15A, 15B, and 15C are cross-sectional views illustrating the ultrasonic probe 100 of FIG. 8 when the ultrasonic probe 100 is seen from the subject 103.

FIGS. 15A, 15B, and 15C illustrate an example in which the ultrasonic reception unit 102 has a plurality of detection elements 102′ and the detection elements 102′ constitute an array probe (102) arrayed at least in the first direction. The light emission unit 101 includes a plurality of light emission end portions 105a and 105b arranged at least along the first direction. FIG. 15A is an example in which light emission end portions 105a and 105b, and acquisition units 104a and 104b are provided on both sides of the array probe (102) arrayed in the first direction. In the example of FIG. 15A, the light beams having the light quantities controlled based on the information about the contact state acquired by the acquisition units 104a and 104b are output from the light emission end portions 105a and 105b. For example, when it is detected that the ultrasonic probe 100 is in the non-contact state as a result of the acquisition unit 104a acquiring the contact state between the subject 103 and the ultrasonic probe 100, the light emitted from the light emission end portion 105a may not be sufficiently emitted on the subject 103. The light quantity of the light emitted from the light emission end portion 105b is increased to supplement the quantity of light that is not emitted from the light emission end portion 105a, whereby the subject 103 can be irradiated with sufficient light.

When the contact state is different between both sides of the array probe arrayed in the first direction, at least part of the light output from the light emission end portion arranged on the side with a weaker contact state may be output from the light emission end portion arranged on the other side.

As illustrated in FIG. 15B, the acquisition unit 104 may be provided on one side of the array probe (ultrasonic reception unit 102). As illustrated in FIG. 15C, the light emission end portions 105a and 105b may be arranged on one side of the array probe (ultrasonic reception unit 102).

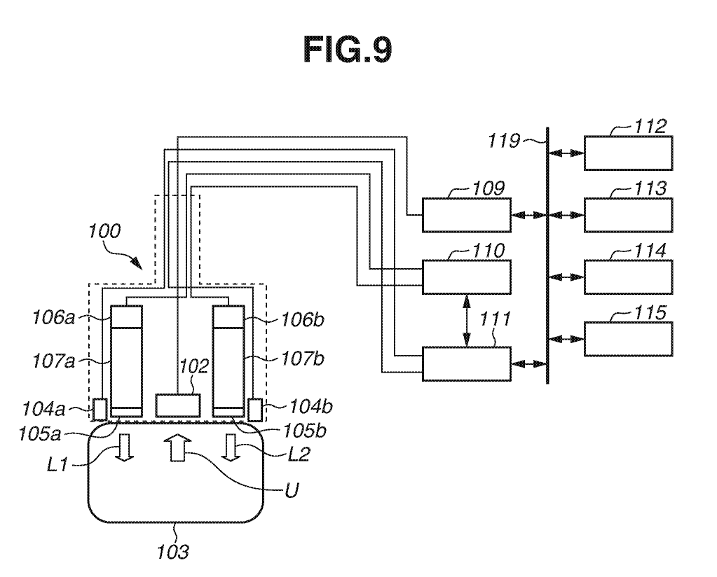

The hand-held ultrasonic probe device according to Example 1 of the present disclosure is illustrated in FIG. 9. In the present exemplary embodiment, the light source array 106 (106a, 106b) is provided on each side of the probe, and the light emitted from the light source is guided to the light emission end portion 105 (105a, 105b) by the light guide path 107. The light source array 106 has at least one of an LED array and an LD array.

After the ultrasonic probe 100 is properly pressed against the subject 103, light is emitted on the subject 103. The light source control unit 110 controls light emission ON/OFF and the light intensity of the light source 106. The acoustic wave generated in the subject receiving the light is received by the ultrasonic reception unit 102 and is output as an electric signal, and then the output signal is sent to the signal processing unit 109 so that information about the subject is acquired. In the case of a device using the photoacoustic effect, the information about the subject to be acquired indicates a source distribution of acoustic waves caused by light irradiation, an initial sound pressure distribution in the subject, a light energy absorption density distribution and an absorption coefficient distribution derived from the initial sound pressure distribution, and a concentration distribution of substances constituting tissues. The concentration distribution of substances is, for example, an oxygen saturation distribution, a total hemoglobin concentration distribution, or an oxidized/reduced hemoglobin concentration distribution.

The ultrasonic probe device according to the present exemplary embodiment further includes an operation unit 114 for allowing a user (mainly an examiner such as a medical worker) to input instructions to the device such as start of imaging and parameters necessary for imaging, and an image configuration unit 113 for imaging the obtained subject information. The ultrasonic probe device according to the present exemplary embodiment further includes a display unit 112 for displaying a UI with which the generated image and the device are operated.

The ultrasonic probe device according to the present exemplary embodiment further includes a control processor 111. The control processor 111 receives various operations of the user via the operation unit 114, generates control information required for generating information about a target subject, and controls each function via a system bus 119. The ultrasonic probe device according to the present exemplary embodiment includes a storage unit 115 that stores information about the acquired photoacoustic wave digital signal, generated images, and other operations.

The ultrasonic probe according to the present exemplary embodiment can adjust the intensity of light emitted on the subject according to the contact state, so that the subject can be irradiated with light of a sufficient light intensity regardless of the contact state with the subject.

The details of each unit constituting the ultrasonic probe according to the present exemplary embodiment will be described below.

### (Light Source)

From the light source 106 according to the present exemplary embodiment, pulsed light of a wavelength absorbed by a specific component of the components constituting the living body is emitted. It is desirable that the wavelength used in the present exemplary embodiment be a wavelength allowing light to propagate to the inside of the subject. More specifically, when the subject is a living body, the wavelength used in the present exemplary embodiment is 600 nm or more and 1100 nm or less. In order to efficiently generate a photoacoustic wave, the pulse width is desirably about 10 to 100 nanoseconds. As a light source, a laser capable of obtaining a large output is desirable, but an LED, a flash lamp, or the like can be used instead of the laser. As the laser, various lasers such as a solid laser, a gas laser, a dye laser, and a semiconductor laser can be used. The time point, waveform, intensity, and the like of irradiation are controlled by the light source control unit. This light source control unit may be integrated with the light source. The light source may be provided separately from the photoacoustic device according to the present exemplary embodiment.

The light source according to the present exemplary embodiment may be a light source capable of emitting light of a plurality of wavelengths.

### (Ultrasonic Reception Unit)

A detection element is arranged in the ultrasonic reception unit 102 according to the present exemplary embodiment, and the detection element outputs a detection signal by detecting a photoacoustic wave occurring in the inside of the living body and on the surface of the living body by the pulsed light. The detection element is for converting a photoacoustic wave into an electric signal. Any detection element may be used as long as it can detect a photoacoustic wave, such as a detection element using a piezoelectric phenomenon, a detection element using a resonance of light, and a detection element using a change of an electrostatic capacity. A PMUT is an example of piezoelectric transducers using a piezoelectric phenomenon. A CMUT is an example of electrostatic capacity type transducers using changes in electrostatic capacity. The CMUT is more desirable as a detection element because it can detect a photoacoustic wave in a wide frequency band.

In order to obtain a high-resolution photoacoustic image, it is desirable to arrange a plurality of detection elements in two dimensions or three dimensions to perform scanning. A reflection film such as a gold film may be provided on the surface of the probe to return the light reflected from the surface of the subject or the holding part, or the light scattered in the inside of the subject and coming out of the subject to the subject again.

### (Signal Processing Unit)

The signal processing unit 109 according to the present exemplary embodiment amplifies the photoacoustic wave signal generated by the ultrasonic reception unit 102 and converts the signal into a photoacoustic wave digital signal which is a digital signal. The signal processing unit 109 according to the present exemplary embodiment includes a signal amplification unit (not illustrated) that amplifies the analog signal generated by the ultrasonic reception unit 102, and an A/D conversion unit (not illustrated) that converts the analog signal into a digital signal.

Furthermore, for the photoacoustic wave digital signal, the signal processing unit 109 according to the present exemplary embodiment performs correction of the sensitivity variation of the ultrasonic reception unit 102 and supplementation processing of the transducer physically or electrically damaged. Further, the signal processing unit 109 can also perform integration processing for noise reduction and the like. The photoacoustic signal obtained by detecting the photoacoustic wave emitted by a light absorption substance inside of the subject 103 is generally a weak signal. By applying integration averaging processing to the photoacoustic wave signals obtained repeatedly at the same position of the subject 103 by the integration processing, system noise can be reduced and the S/N ratio of the photoacoustic wave signal can be improved.

### (Control Processor)

The control processor 111 according to the present exemplary embodiment runs an OS to control and manage basic resources in the program operation, reads the program code stored in the storage unit 115, and executes the functions described below. The control processor 111 receives event notifications generated by various operations such as start of imaging from the user via the operation unit 114, manages acquisition operation of the subject information, and controls each piece of hardware via the system bus 119. The control processor 111 further commands the light source control unit 110 to control the light source 106 required for generating the subject information of interest. An example of the control processor is a CPU.

### (Operation Unit)

The operation unit 114 according to the present exemplary embodiment is an input device for the user to perform parameter setting related to imaging of, for example, the visualization range of the subject information, instruction to start imaging, and other image processing operations related to images. In general, the operation unit 114 according to the present exemplary embodiment is constituted by a mouse, a keyboard, a touch panel, and the like, and performs event notification to the software such as the OS operating on the control processor 111 according to the operation of the user.

### (Image Configuration Unit)

Based on the acquired photoacoustic wave digital signal, the image configuration unit 113 according to the present exemplary embodiment converts tissue information within the subject into an image and constructs a display image such as an arbitrary tomographic image of the photoacoustic wave image. Various correction processes such as luminance correction, distortion correction, and ROI cropping are applied to the constructed image to construct more desirable information for diagnosis. According to the operation of the user via the operation unit 114, parameters related to the configuration of the photoacoustic wave image are adjusted, and display images are adjusted. The photoacoustic wave image is obtained by performing image reconstruction processing on the digital signal of the three-dimensional photoacoustic wave generated from the ultrasonic reception unit 102. The photoacoustic wave image can visualize a characteristic distribution such as acoustic impedance and subject information such as an optical characteristic value distribution. For example, back projection in time domain or Fourier domain commonly used in tomography technology, or phasing addition processing is used for the image reconstruction processing. The image configuration unit 113 is generally constructed using a GPU having a high-performance arithmetic operation processing function and a graphic display function. This can shorten the time taken to execute the image reconstruction processing and to configure a display image.

### (Display Unit)

The display unit 112 according to the present exemplary embodiment displays a photoacoustic wave image generated by the image configuration unit 113 and a UI for operating an image and the device. For example, a liquid crystal display is used, but any type of display such as organic EL may be used.

### (Storage Unit)

The storage unit 115 according to the present exemplary embodiment includes a memory required for the control processor 111 to operate, and a memory temporarily holding data in the subject information acquisition operation. Furthermore, the storage unit 115 includes a storage medium such as a hard disk that stores and holds the generated photoacoustic wave image, relevant subject information, and diagnostic information. The storage unit 115 stores the program code of software that realizes the function described below.

### (Subject (Optical Absorber))

The subject (optical absorber) does not form part of the ultrasonic probe according to the present exemplary embodiment, but the subject (optical absorber) will be described below. The main purposes of the ultrasonic probe according to the present exemplary embodiment using the photoacoustic effect include imaging blood vessels, diagnosis of human and animal malignant tumor and vascular disease, and follow-up of chemotherapy. The optical absorber inside of the subject has an absorption coefficient relatively high within the subject depending on the wavelength of the light to be used. More specifically, the optical absorber inside of the subject may be water, fat, protein, oxidized hemoglobin, reduced hemoglobin, and the like.

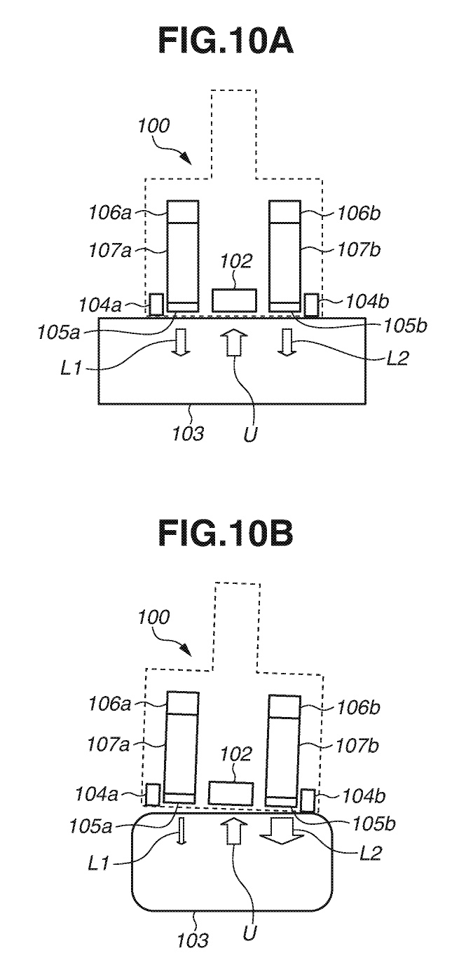

The problems associated with hand-held ultrasonic probes are illustrated in FIGS. 10A and 10B. FIG. 10A illustrates a state in which there is no gap between the ultrasonic probe and the subject, and the ultrasonic probe and the subject are in close contact with each other to be pressed correctly. A jelly-like acoustic coupling agent (acoustic matching agent) is generally applied between the ultrasonic probe 100 and the subject 103. The space between the probe and the body is filled with a jelly-like substance having a unique acoustic impedance close to the living body (the product of sound speed and density), whereby mixing of air can be avoided, and sound waves can easily propagate between the probe 100 and the subject 103. Therefore, a certain degree of probe inclination (clearance between the ultrasonic probe 100 and the subject 103) is permitted at the time of inspection.

When the ultrasonic probe 100 is in close contact with the subject 103, the light L1 emitted from the light emission end portion 105a and the light L2 emitted from the light emission end portion 105b can be made incident on the subject 103 in such a manner that the light quantities of the light L1 and the light L2 are substantially uniform. As a result, the signal level of the ultrasonic wave U from the subject 103 can be kept properly.

However, as illustrated in FIG. 10B, when the inclination exceeds the allowable value and the subject 103 and the ultrasonic probe 100 are in the non-contact state, air can enter the gap between the probe 100 and the subject 103. As a result, the light quantity of the light L1 emitted from the side where the gap is formed (light emission end portion 105a) decreases. This leads to a decrease in the total quantity of light incident on the subject 103. As a result, the signal level of the ultrasonic wave U decreases, and thus a correct photoacoustic image may not be obtained.

Whether the ultrasonic probe 100 according to the present exemplary embodiment is closely pressed against the subject 103 in parallel can be determined from information given by the acquisition units 104a and 104b, which acquire the information about the contact state, provided on both side surfaces of the ultrasonic probe 100. The acquisition unit 104 should at least be a unit that detects formation of a gap at the distal end of the probe, such as a mechanical switch, a load sensor, or a displacement sensor.

Subsequently, the operation to keep the light quantity proper will be described. As illustrated in FIG. 9, the signals from the acquisition units 104a and 104b that acquire information about the contact state are taken into the control processor 111, the contact state is detected, and an arithmetic operation about in which direction the probe 100 is inclined is performed, for example. As illustrated in FIG. 10B, when it is detected that one acquisition unit 104b is correctly in contact with the subject 103 and there is a gap from the other acquisition unit 104a, a command is given to the light source control unit 110 to increase the output of the light source 106b.

Depending on the shape of the surface of the subject 103, the ON/OFF state of the contact state may be frequently switched, but this can be solved by providing a hysteresis circuit in the control processor 111 that receives the signal from the acquisition unit 104. The hysteresis circuit prevents frequent switching. In the case of a subject which cannot be handled even by the hysteresis circuit, this can be solved by providing a function of acquiring the photoacoustic signal by canceling the adjustment function of the light quantity of the light source to keep the light quantity constant.

As described above, when the subject 103 is irradiated by adjusting the outputs of the left and right light sources 106a and 106b, the image configuration unit 113 is provided with a table referring to the quantity (distribution) of light to be emitted on the surface of the subject 103 according to the light quantity adjustment. The light quantities from the right and left light sources are read from the table, and the light intensity distribution inside the living body is calculated. The quantitative information (oxygen saturation distribution, total hemoglobin concentration distribution, oxidized/reduced hemoglobin concentration distribution) and the like of the received signal are calculated from the light intensity distribution, and are displayed on the display unit 112.

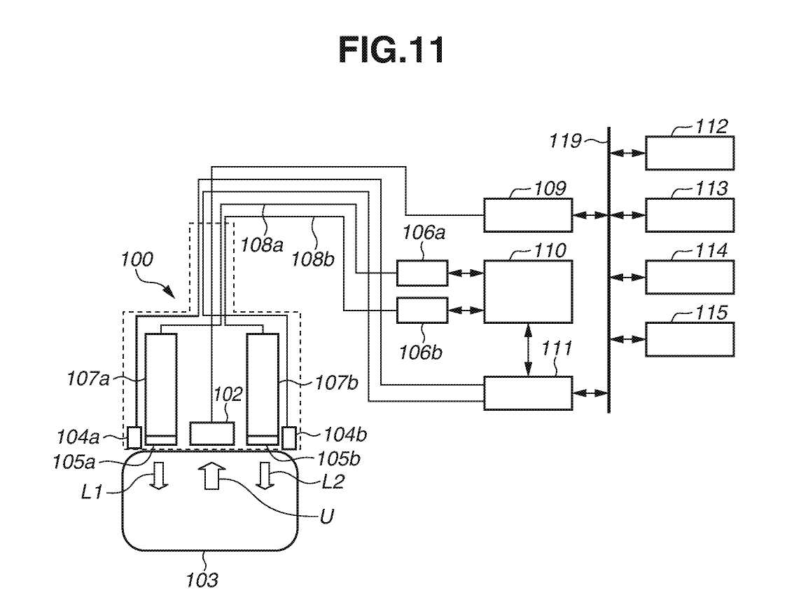

What should be noted here is that when only the output of one light source is increased, heat of the light source may be trapped inside the probe 100, electrical parts may break down, and the subject (examinee) may be burned. FIG. 11 illustrates an ultrasonic probe provided with a wave guide unit 108 for guiding light from an externally provided light source 106 to the light emission end portion. An example of the wave guide unit 108 is an optical fiber. When the light source is provided externally, there are advantages such as easy thermal design obtained by keeping the heat source away from the probe 100 and easy maintenance of the light source.

According to this configuration, the light quantity of the light L2 can be raised to compensate for the decrease in the light quantity of the light L1, the quantity of light incident on the subject can be ensured, and the signal level of the ultrasonic wave U from the subject 103 is maintained properly, so that high quality images can be obtained.

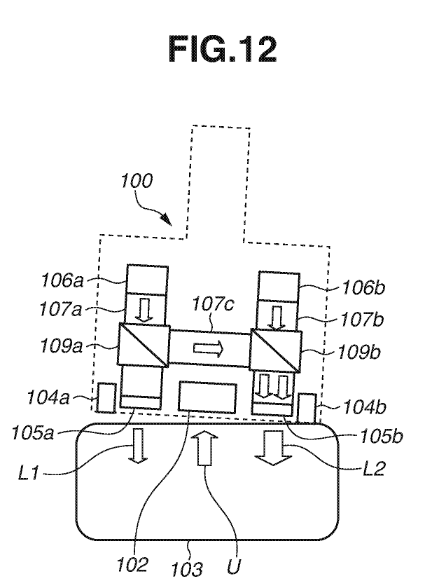

A hand-held ultrasonic probe according to Example 2 of the present disclosure is illustrated in FIG. 12. An ultrasonic reception unit 102 is provided in the ultrasonic probe 100. Light sources 106a and 106b, light guide paths 107a and 107b, and light emission end portions 105a and 105b are provided on both sides of the ultrasonic reception unit 102. Further, a light guide path 107c connecting the light guide paths on both sides is provided, and light guide path switch units 116 (116a, 116b) are configured to switch the light paths from the light sources to the light emission end portions on both sides.

Subsequently, operation according to the present exemplary embodiment for properly maintaining the light quantity of the light emitted from the ultrasonic probe 100 toward the subject will be described. As illustrated in FIG. 12, the contact state is detected based on signals from the acquisition units 104a and 104b that acquire information about the contact state, and an arithmetic operation of, for example, the inclination direction as to which side the probe 100 is inclined is performed. As illustrated in FIG. 12, for example, an operation will be described in a case where it is detected that one acquisition unit 104b is correctly in contact with the subject 103 and there is a gap from the other acquisition unit 104a.

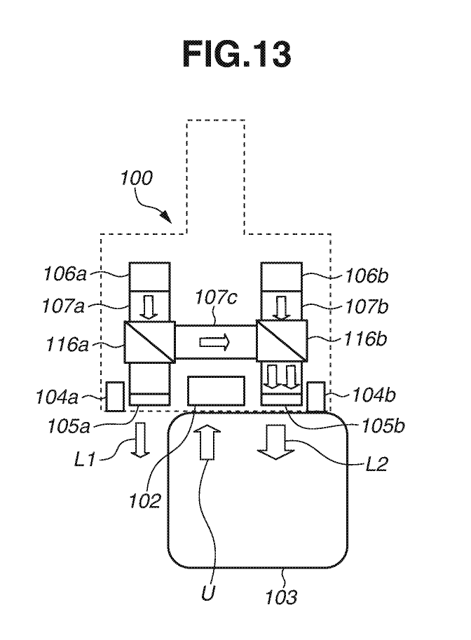

In this case, since the gap is formed between the probe 100 and the subject 103, the light from the light emission end portion 105a is greatly attenuated. Therefore, the light guide path switch unit 116 is appropriately switched by a control processor (not illustrated), and the light from the light source 106a on the side where the probe 100 is not in contact with the subject is guided via the light guide path 107c to the light emission end portion 105b on the side where the probe 100 is in contact with the subject. If the inclination of the probe is opposite to the example above, the light guide path switch unit 116 is used to switch the light guide path appropriately, and the light from the light source 106b can be guided to the light emission end portion 105a via the light guide path 107c. In this example, unlike Example 1 described above, it is not necessary to increase the output of one light source to ensure a light quantity, and therefore the heat generated inside the probe 100 is less likely to increase. Therefore, as illustrated in FIG. 13, when a subject with high curvature (such as an arm) is to be observed, light from one light source can be guided to the other light emission end portion even if only one of the light emission end portions 105 can contact the subject, resulting in an advantage in that a light quantity for the subject can be ensured.

In the present exemplary embodiment, for example, a case where all the light from one light source is guided to the other light emission end portion has been described, but a light adjusting device can be used as the light guide path switch unit 116. When the light adjusting device is used, only part of the light can be guided to the other light emission end portion, and the light quantity can be distributed in a stepwise manner according to the contact state.

According to this configuration, the light from the light source 106a on the side not in contact with the subject can be emitted from the light emission end portion on the side in contact with the subject. As a result, the quantity of light incident on the subject can be ensured, and a high quality image about the subject can be obtained by maintaining the signal level of the ultrasonic wave U from the subject 103 properly.

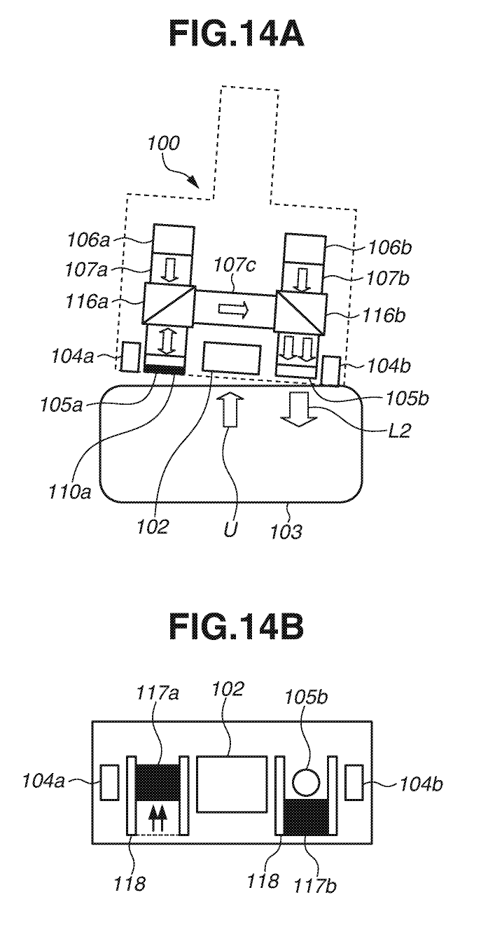

A hand-held ultrasonic probe according to Example 3 of the present disclosure is illustrated in FIG. 14A. A view of the hand-held ultrasonic probe according to Example 3 of the present disclosure as seen from the subject side is illustrated in FIG. 14B. Light sources 106a and 106b, light guide paths 107a and 107b, and light emission end portions 105a and 105b are provided on both sides of the probe. In addition, a light guide path 107c connecting the light guide paths on both sides is provided, and a light guide path switch unit 116 is used to switch the light paths from the light sources to the light emission end portions on both sides.

Subsequently, the operation for keeping the light quantity proper will be described. As illustrated in FIGS. 14A and 14B, the contact state is detected based on signals from the acquisition units 104a and 104b that acquire information about the contact state, and an arithmetic operation of, for example, the inclination direction as to which side the probe 100 is inclined is performed. As illustrated in FIGS. 14A and 14B, for example, an operation will be described in a case where it is detected that one acquisition unit 104b is correctly in contact with the subject 103 and there is a gap from the other acquisition unit 104a.

In this case, since the gap is formed between the probe 100 and the subject 103, the light from the light emission end portion 105a is greatly attenuated. Therefore, a shutter 117 supported by a shutter guide part 118 is provided in the light emission end portion 105a, and the shutter is driven by a controller (not illustrated) to cut the light emitted from the light emission end portion 105a to the subject such that the light on the side where the gap is formed does not leak to the outside. Furthermore, a mirror or a corner cube for reflecting light is provided on the upper surface (the ultrasonic probe side) of the shutter, so that the light from the light source 106a can be guided to the opposite light emission end portion 105b by appropriately switching the light guide path switch unit 116.

According to this configuration, the light from the light source 106a on the side not in contact with the subject can be blocked not to leak to the outside, and the safety can be improved. Further, the light emission end portion on the side that is in contact with the subject can emit light. As a result, the quantity of light incident on the subject can be ensured, and a high quality image can be obtained by properly maintaining the signal level of the ultrasonic wave U from the subject 103.

The description of the reference numerals in the description of the second exemplary embodiment and FIGS. 8 to 15 will be given below.
100 ultrasonic probe
101 light emission unit
102 ultrasonic reception unit
103 subject
104a, 104b acquisition unit
105a, 105b light emission end portion
106a, 106b light source
107a, 107b, 107c light guide path
108a, 108b wave guide unit
109 signal processing unit
110 light source control unit
111 control processor
112 display unit
113 image configuration unit
114 operation unit
115 storage unit
116a, 116b light guide path switch unit
117a, 117b shutter
118 shutter guide part
119 system bus

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Applications No. 2016-229309, filed Nov. 25, 2016, and No. 2016-229310, filed Nov. 25, 2016, which are hereby incorporated by reference herein in their entirety.

What is claimed is:

1. A hand-held ultrasonic probe comprising:
a grip unit;
a light irradiation unit; and
an ultrasonic reception unit,
wherein the light irradiation unit includes:
a change unit configured to change a light intensity distribution of light emitted on a subject; and
a reception unit configured to receive an instruction from a user about a change in the light intensity distribution of the light from the light irradiation unit in a state in which the user grips the ultrasonic probe, and
wherein the light irradiation unit obtains a signal for controlling the change unit based on the instruction from the user received by the reception unit, and drives the change unit using the signal to change the light intensity distribution of the light for the subject.

2. The hand-held ultrasonic probe according to claim 1, comprising, as the reception unit, a contact sensor configured to acquire information about a contact state between the subject and the ultrasonic probe,
wherein the instruction from the user is given via the contact sensor.

3. The hand-held ultrasonic probe according to claim 1, comprising, as the reception unit, a pressure sensor in the grip unit,
wherein the instruction from the user is given via the pressure sensor.

4. The hand-held ultrasonic probe according to claim 1, comprising, as the reception unit, a switch unit in the ultrasonic probe,
wherein the instruction from the user is given via the switch unit.

5. The hand-held ultrasonic probe according to claim 1, wherein the change unit is configured to change an irradiation position of light on the subject from the light irradiation unit.

6. The hand-held ultrasonic probe according to claim 1, wherein the change unit is configured to change an irradiation angle of light on the subject from the light irradiation unit.

7. The hand-held ultrasonic probe according to claim 1, wherein a light source array is embedded in the ultrasonic probe as the light irradiation unit.

8. The hand-held ultrasonic probe according to claim 1, wherein the light irradiation unit is provided with a wave guide unit configured to guide light from an externally provided light source to a light emission end portion.

9. A hand-held ultrasonic probe comprising:
a light emission unit configured to emit light on a subject;
an ultrasonic reception unit configured to receive an ultrasonic wave generated by emission of the light on the subject; and
an acquisition unit configured to acquire information about a contact state between the subject and the ultrasonic probe,
wherein the light emission unit includes a plurality of light emission end portions of which output light quantities are independently controlled, and
wherein light with light quantities controlled based on the information about the contact state is output from the plurality of light emission end portions.

10. The hand-held ultrasonic probe according to claim 9,
wherein the ultrasonic reception unit includes an array probe in which a plurality of detection elements is arrayed at least in a first direction, and
wherein the light emission unit includes the plurality of light emission end portions arranged at least in the first direction.

11. The hand-held ultrasonic probe according to claim 10,
wherein the light emission end portions are provided on both sides of the array probe arrayed in the first direction, and
wherein light with light quantities controlled based on the information about the contact state acquired by the acquisition unit is output from the light emission end portions arranged on both sides of the array probe.

12. The hand-held ultrasonic probe according to claim 9, wherein the acquisition unit is configured to acquire the information about the contact state using a load sensor or a displacement sensor.

13. The hand-held ultrasonic probe according to claim 9, wherein the acquisition unit is configured to acquire information about an inclination of the ultrasonic probe with respect to the subject.

14. The hand-held ultrasonic probe according to claim 10, wherein the acquisition unit is configured to acquire the contact state with the subject on each of both sides of the array probe arrayed in the first direction.

15. The hand-held ultrasonic probe according to claim 14, wherein in a case where the contact state is different between both sides of the array probe arrayed in the first direction, at least part of light output from the light emission end portion arranged on a side where the contact state is weaker is to be output from the light emission end portion arranged on the other side.

16. The hand-held ultrasonic probe according to claim 9, wherein, in the light emission unit, a light source array is embedded in the ultrasonic probe.

17. The hand-held ultrasonic probe according to claim 9, wherein the light emission unit is provided with a wave guide unit configured to guide light from an externally provided light source to the light emission end portion.

18. The hand-held ultrasonic probe according to claim 9,
wherein the ultrasonic reception unit is configured to receive an ultrasonic wave and output an electric signal, and
wherein information about the subject is acquired based on the electric signal.

19. A system comprising:
the hand-held ultrasonic probe according to claim 9; and
a processing unit configured to perform control based on the information about the contact state.
